(19) United States
(12) Patent Application Publication
(10) Pub. No.: US 2023/0377692 A1
(43) Pub. Date: Nov. 23, 2023

(54) METHODS AND SYSTEMS FOR STORING GENOMIC DATA IN A FILE STRUCTURE COMPRISING AN INFORMATION METADATA STRUCTURE

(71) Applicant: KONINKLIJKE PHILIPS N.V., EINDHOVEN (NL)

(72) Inventor: Yee Him Cheung, Boston, MA (US)

(21) Appl. No.: 18/028,222

(22) PCT Filed: Oct. 4, 2021
(86) PCT No.: PCT/EP2021/077298

§ 371 Date: Mar. 24, 2023

Related U.S. Application Data
(60) Provisional application No. 63/088,055, filed on Oct. 6, 2020.

(30) Foreign Application Priority Data
Oct. 6, 2020 (EP) 63088055

(51) Int. Cl. G16B 50/50; G16B 50/40; G16B 50/30; G16B 30/00; G06F 21/62

(52) U.S. Cl. CPC G16B 50/50; G16B 50/40; G16B 50/30; G16B 30/00; G06F 21/6245; G06F 2221/2101

(57) ABSTRACT

A method (100) for storing genomic data within a data structure comprising a file structure, comprising: (i) receiving (120) a genomic dataset comprising a plurality of fields or attributes of different data types; (ii) generating (130) an information metadata structure for the genomic dataset, comprising one or more of: information about an annotation table, including one or more user profiles and associated profile permission; analytics information configured to facilitate verification of data reproducibility; access history for the genomic dataset, configured to facilitate data traceability; and linkage information defining a relationship between the annotation table and one or more data objects; (ii) compressing (140) the genomic data and information metadata using a compression algorithm; and (iv) storing (150) the compressed genomic dataset and information metadata in a container data structure; wherein some or all of the annotation table is encrypted.

### FIELD OF THE DISCLOSURE

The present disclosure is directed generally to methods and systems for storing large quantities of data with associated metadata, and, in particular, to the compression and storage of genomic data.

### BACKGROUND

High-throughput genomic sequencing (HTS) is an important tool for genomics research, and has numerous applications for discovery, diagnosis, and other methodologies. Often, the results of HTS are processed further to obtain higher-level information. The process of aggregating information deduced from single reads and their alignments to the genome into more complex results is generally known as secondary analysis. In most HTS-based biological studies, the output of secondary analysis is usually represented as different types of annotations associated to one or more genomic intervals on the reference sequences.

Indeed, biological studies typically produce genomic annotation data such as mapping statistics, quantitative browser tracks, variants, genome functional annotations, gene expression data and Hi-C contact matrices. These diverse types of downstream genomic data are currently represented in different formats such as VCF, BED, WIG, and many, many more. These formats typically comprise loosely defined semantics, which leads to issues with interoperability, the need for frequent conversions between formats, difficulty in the visualization of multi-modal data, and complicated information exchange, among other issues.

Additionally, the lack of a single format for diverse types of genomic annotation data has stifled work on compression algorithms and has led to the widespread use of general compression algorithms with suboptimum performance. These algorithms do not exploit the fact the annotation data typically comprises of multiple fields (attributes) with different statistical characteristics and instead compress them together. Further, these prior art storage mechanisms lack functional metadata for supporting advanced features such as data security and privacy, authenticity, access tracking, reproducibility verification, data linkages, and profile management.

### SUMMARY OF THE DISCLOSURE

There is a continued need for a unified data format for the efficient representation and compression of diverse genomic annotation data for file storage and data transport. There is a further need for associating and storing metadata with the compressed genomic data to enable data security and privacy, authenticity, access tracking, reproducibility verification, data linkages, and profile management, among other advantages.

The present disclosure is directed to inventive methods and systems for storing genomic data within a data structure comprising a file structure, together with functional metadata integrated into the file structure. Various embodiments and implementations herein are directed to a system or method that receives genomic data and stores that genomic data within a data structure comprising a file structure. The genomic data can be any of a wide variety of different genomic data types, including but not limited to genomic variants (VCF), gene expressions, genomic functional annotations (e.g., BED, GTF, GFF, GFF3, GenBank, etc.), quantitative browser tracks (e.g., Wig, BigWig, BedGraph, etc.), and/or chromosome conformation capture (e.g., HiC files, etc.), among many others. Information metadata to accompany the genomic dataset is generated and stored with the genomic data file structure. The information metadata comprises one or more of: (i) information about the annotation table within the file structure, including one or more user profiles and associated profile permissions; (ii) analytics information detailing a source dataset and one or more processing steps for producing the genomic dataset, wherein the analytics information is configured to facilitate verification of data reproducibility; (iii) access history for the genomic dataset, configured to facilitate data traceability; and (iv) linkage information defining a relationship between the annotation table and one or more data objects, wherein the linkage information is configured to enhance data navigation and/or to support data queries across the linked data. The genomic data is compressed, and the information metadata is compressed, using one or more compression algorithms to generate a compressed genomic dataset and compressed information metadata. The compressed genomic dataset and the compressed information metadata is then stored in a container data structure.

Generally, in one aspect, a method for storing genomic data within a data structure comprising a file structure is provided. The method includes: receiving a genomic dataset comprising a plurality of fields or attributes of different data types; generating an information metadata structure for the genomic dataset, comprising one or more of: (i) information about an annotation table within the file structure, including one or more user profiles and associated profile permission; (ii) analytics information detailing a source dataset and one or more processing steps for producing the genomic dataset, wherein the analytics information is configured to facilitate verification of data reproducibility; (iii) access history for the genomic dataset, configured to facilitate data traceability; and (iv) linkage information defining a relationship between the annotation table and one or more data objects, wherein the linkage information is configured to enhance data navigation and/or to support a data query across linked data; compressing the genomic data, and the information metadata, using one or more compression algorithms to generate a compressed genomic dataset and compressed information metadata; and storing the compressed genomic dataset and the compressed information metadata in a container data structure; wherein some or all of the annotation table is encrypted.

According to an embodiment, the method further includes receiving new data for the annotation table; and updating the annotation table with the new data, comprising updating one or both of the information metadata and the genomic data.

According to an embodiment, one or more of (i) through (iv) comprise selective encryption and a digital signature.

According to an embodiment, the access history for the genomic dataset is configured to track access and/or change to the genomic data by one or more users, and wherein tracked access or changes are predefined.

According to an embodiment, the access history further comprises an identity of a user that accessed the genomic data and/or made a change to the genomic data, and wherein the access history optionally comprises an accompany digital signature for the user.

According to an embodiment, the one or more user profiles comprise one or more parameters for presentation and/or further processing such as filtering, sorting, and/or highlighting of the genomic data.

According to an embodiment, the one or more user profiles can be created by a user, encrypted for confidentially, signed for authenticity, and/or shared with another designated user.

According to an embodiment, the analytics information comprises instructions for verification of data reproducibility by evaluating a concordance of the genomic dataset with an existing counterpart genomic dataset being verified.

According to an embodiment, the analytics information further comprises one or more verification results, with an optional digital signatures by a user that performed the verification.

According to an embodiment, the linkage information comprises one or more specifications for mapping data between one or more annotation tables.

According to an embodiment, the method further comprises verifying data reproducibility using the analytics information and authenticity and/or integrity of the access history.

According to a second aspect is a system for storing genomic data within a data structure comprising a file structure. The system includes: a genomic dataset comprising a plurality of fields or attributes of different data types; a container data structure configured to store compressed genomic data and compressed information metadata; a data compression algorithm; and a processor configured to: (i) generate an information metadata structure for the genomic dataset, comprising one or more of: (1) information about an annotation table within the file structure, including one or more user profiles and associated profile permission; (2) analytics information detailing a source dataset and one or more processing steps for producing the genomic dataset, wherein the analytics information is configured to facilitate verification of data reproducibility; (3) access history for the genomic dataset, configured to facilitate data traceability; and (4) linkage information defining a relationship between the annotation table and one or more data objects, wherein the linkage information is configured to enhance data navigation and/or to support a data query across linked data; (ii) compress, using the data compression algorithm, the genomic data and the information metadata to generate a compressed genomic dataset and compressed information metadata; and (iii) store the compressed genomic dataset and the compressed information metadata in a container data structure; wherein some or all of the annotation table is encrypted.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for packaging genomic data, in accordance with an embodiment.

FIG. 2 is a schematic representation of a genomic data storage system, in accordance with an embodiment.

FIG. 3 is a schematic representation of a data file structure, in accordance with an embodiment.

### DETAILED DESCRIPTION OF EMBODIMENTS

The present disclosure describes various embodiments of a system and method for storing genomic data and associated information metadata within a data structure. Applicant has recognized and appreciated that it would be beneficial to provide a method and system comprising a unified data format for the efficient representation and compression of diverse genomic annotation data. A genomic data storage system receives a genomic dataset comprising a plurality of fields or attributes of different data types. The system generates information metadata for the genomic dataset. The information metadata comprises one or more of: (i) information about an annotation table, including one or more user profiles and associated profile permissions; (ii) one or more parameters configured to facilitate verification of data reproducibility; (iii) access history for the genomic dataset, configured to facilitate data traceability; and (iv) one or more linkages between the annotation table and one or more data objects. The genomic data and information metadata is compressed using one or more compression algorithms, and the compressed data is then stored in memory.

Extending a metadata and security framework with stored genomic data provides advanced functionalities for enhancing the management and analysis of the data, which is especially important for large-scale collaborative genomic studies. For example, the methods and systems described or otherwise envisioned herein enables selective encryption and digital signature(s) to be applied only to sensitive information as decided by users, thereby reducing the computational burden and processing overhead for the enforcement of data security and privacy. The methods and systems further enable non-repudiable access tracking for data traceability such that selected operations and changes to the data can be traced and accounted for. They also allow for automatic verification and proof of data reproducibility critical for applications such as scientific studies, manuscript publications, and clinical applications. The methods and systems allow for the establishment of data linkages to specify relationships between data objects for enhancing functions such as data exploration, navigation, visualization, and join query. Further, they enable the management of view profiles that contain parameters for the presentation, filtering, sorting, and highlighting of annotation table data. Another key advantage of integrating functional metadata into the overall file format is that such crucial metadata is organized and readily available as part of the data file, and is not easily lost or misplaced during data transfer and migration. Further, since data security and privacy is designed into the file format rather than being offered through the storage platform or file management software, stronger data protection is achieved. Moreover, with the syntax and processing mechanism of the information and protection metadata clearly defined in the standard, users can expect consistent or similar functionalities and performance from any compliant software.

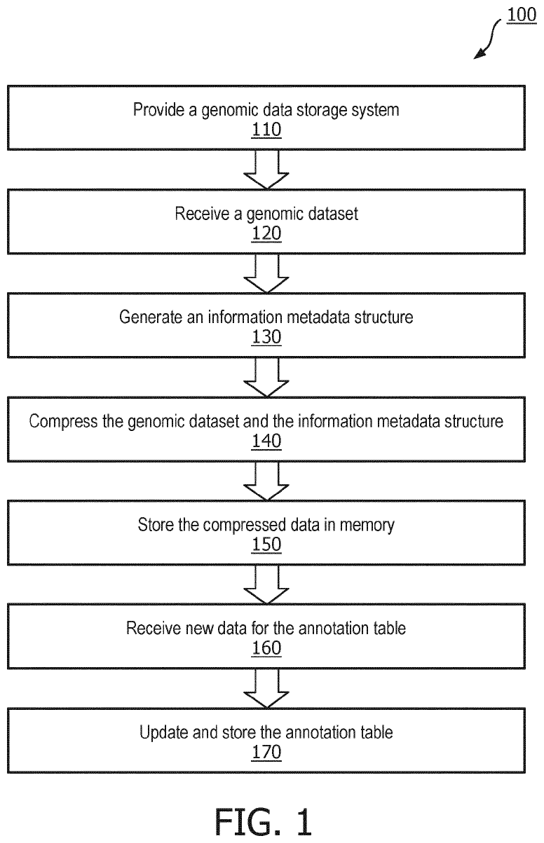

Referring to FIG. 1, in one embodiment, is a flowchart of a method 100 for storing genomic data and associated information metadata within a data structure comprising a file structure using a genomic data storage system. The methods described in connection with the figures are provided as examples only, and shall be understood not limit the scope of the disclosure. The genomic data storage system can be any of the systems described or otherwise envisioned herein. The genomic data storage system can be a single system or multiple different systems.

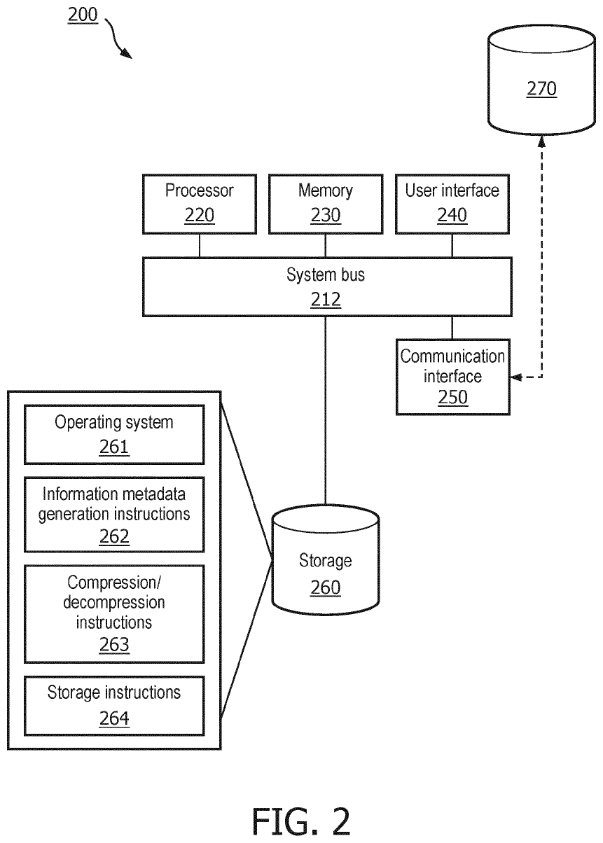

At step 110 of the method, a genomic data storage system is provided. Referring to an embodiment of a genomic data storage system 200 as depicted in FIG. 2, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the system 200 may be different and more complex than illustrated. Additionally, genomic data storage system 200 can be any of the systems described or otherwise envisioned herein. Other elements and components of genomic data storage system 200 are disclosed and/or envisioned elsewhere herein.

At step 120 of the method, the genomic data storage system receives a genomic dataset comprising genomic data with a plurality of fields or attributes of different data types. The genomic data can be any of a wide variety of different genomic data types, including but not limited to genomic variants (VCF), gene expressions, genomic functional annotations (e.g., BED, GTF, GFF, GFF3, GenBank, etc.), quantitative browser tracks (e.g., Wig, BigWig, BedGraph, etc.), and/or chromosome conformation capture (e.g., HiC files, etc.), among many others. The received genomic dataset may comprise genomic data of one type or a plurality of different types of genomic data and/or a plurality of fields or attributes of different data types. The received genomic dataset may utilized immediately for subsequent steps of the methods described or otherwise envisioned herein, or may be stored for future use by this and other methods. Accordingly, the system may comprise or be in communication with local or remote data storage configured to store the genomic dataset.

At step 130 of the method, the genomic data storage system generates an information metadata structure for the genomic dataset. The information metadata structure is configured to enable a wide variety of functionalities, including one or more of support for selective encryption and digital signatures, data traceability or non-repudiable access tracking, verification of data reproducibility, and establishment of linkages between data objects, among other functionalities.

According to an embodiment, the information metadata structure comprises information about an annotation table within the file structure, including one or more user profiles and associated profile permissions. According to an embodiment, the information metadata structure comprises one or more parameters configured to facilitate verification of data reproducibility. According to an embodiment, the information metadata structure comprises access history for the genomic dataset, configured to facilitate data traceability. According to an embodiment, the information metadata structure comprises one or more linkages between the annotation table and one or more data objects configured to enhance data navigation and/or to support a data query across linked data.

The generated information metadata structure may be utilized immediately for subsequent steps of the methods described or otherwise envisioned herein, or may be stored for future use by this and other methods. Accordingly, the system may comprise or be in communication with local or remote data storage configured to store the genomic dataset, annotation table, and/or information metadata structure. Notably, some or all of the information metadata structure may be encrypted as described or otherwise envisioned herein.

At step 140 of the method, the genomic data storage system compresses the genomic data, together with the generated information metadata structure, using a compression algorithm to generate a compressed genomic dataset. The compression algorithm can be any algorithm, method, or process for data transformation and compression, including but not limited to the compression algorithms and methods described or otherwise envisioned herein. The data may be compressed by a single compression algorithm or by multiple compression algorithms.

At step 150 of the method, the compressed genomic dataset, together with the compressed information metadata, is stored in memory in a container data structure. The memory may be any memory capable of receiving and storing the compressed data. The memory may be associated with the genomic data storage system, or may be in direct or indirect wired and/or wireless communication with the genomic data storage system. The memory may be a local or a remote memory. The memory may be a cloud-based memory. Many other storage mechanisms and devices are possible.

At step 160 of the method, the genomic data storage system receives new data for the annotation table. The new data may be provided to the system, may be requested by the system, or is otherwise given to or received by the system. The new data is any data that requires an update of the annotation table. For example, the new data may comprise any one or more of profile or permission modifications or updates, data reproducibility parameters, access information, and/or linkage information between the annotation table and one or more data objects within the genomic data, among a wide variety of other data or information. The new data or information may be processed or otherwise prepared by the genomic data storage system for updating the annotation table. The new data or information may be utilized immediately for subsequent steps of the methods described or otherwise envisioned herein, or may be stored for future use by this and other methods.

At step 170 of the method, the genomic data storage system updates the annotation table with the new data or information, including both the information metadata and the genomic data. The system may retrieve the annotation table and decompress the table using a decompression and/or inverse transform algorithm, which can be any algorithms, methods, or processes for data decompression and inverse transformation. The system can then update the annotation table, and then can compress and store the updated file in memory.

Genomic Data Storage Structure and Data Format

The genomic data storage structure in which the received genomic data and associated annotation table is packaged may take any of a wide variety of formats. Although a specific format is described with reference to an embodiment, below, it is understood that this is just one example of a data structure that may be utilized by the genomic data storage system described or otherwise envisioned herein. Similarly, the format of the data within the genomic data storage structure may take any of a wide variety of formats. Although a specific format is described with reference to an embodiment, below, it is understood that this is just one example of a data format that may be utilized by the genomic data storage system described or otherwise envisioned herein.

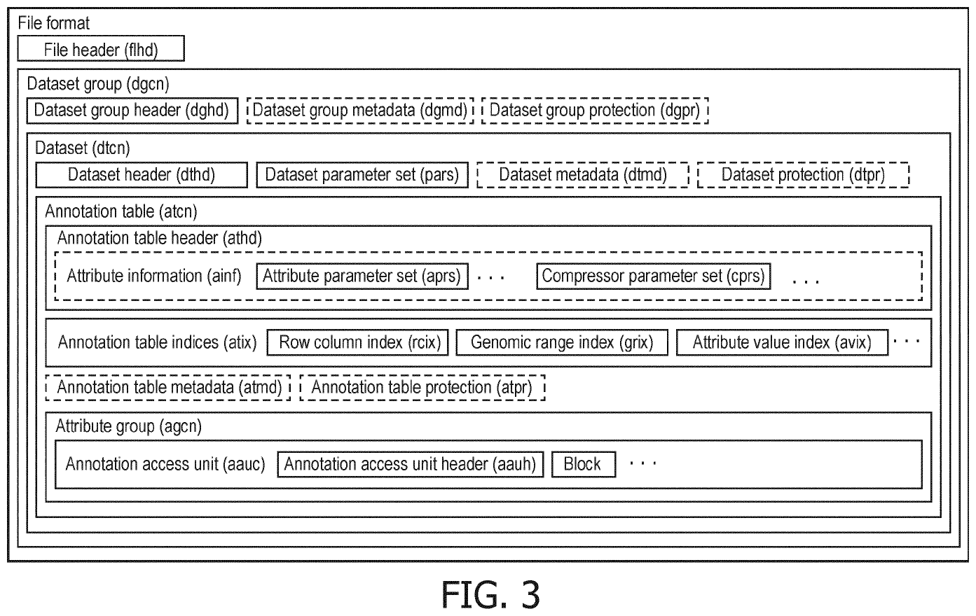

Referring to FIG. 3 is an embodiment of a top-level container hierarchy for a genomic dataset and associated annotation table. In this format, the top-level container boxes of File, Dataset Group, and Dataset are utilized. The Dataset comprises an Annotation Table (atcn) with the data. In FIG. 3, all container boxes, including Dataset Group (dgcn), Dataset (dtcn), Annotation Table (atcn), Attribute Group (agcn), and Annotation Access Unit (aauc), can exist in multiple instances. For example, the “ . . . ” symbol behind a box indicates there can be multiple instances of that specific box structure.

According to an embodiment, the information and protection metadata can be stored respectively in the Annotation Table Metadata and Annotation Table Protection data structures, which are enclosed in gen_info boxes in KLV (Key, Length, Value) format with syntax as follows, although other syntax is possible:

| struct gen_info { | |
|---|---|
| c(4) | Key; |
| u(64) | Length; |
| u(8) | Value[ ]; |

}

According to an embodiment, the Key field specifies the type of the data structure in a four-character code, which is “atmd” for Annotation Table Metadata and “atpr” for Annotation Table Protection. The Length field specifies the number of bytes composing the entire gen_info structure, including all three fields Key, Length and Value. The syntaxes of the Value fields of Annotation Table Metadata and Annotation Table Protection are defined respectively in TABLE 1 and TABLE 2.

TABLE 1
Syntax of Annotation Table Metadata

| Syntax | Key | Type | Remarks |
|---|---|---|---|
| annotation_table_metadata { | atmd | | |
| dataset_group_ID | | u(8) | Dataset group identifier |
| dataset_ID | | u(16) | Dataset identifier |
| AT_ID | | u(8) | Annotation table identifier |
| ATMD_general_exist | | u(1) | Flag for the existence of general |
| | | | information |
| if (ATMD_general_exist) { | | | |
| ATMD_general_size | | u(16) | Size in number of bytes of general |
| | | | information |
| ATMD_general( ) | | u(v) | General information of the annotation |
| | | | table |
| } | | | |
| ATMD_analytics_exist | | u(1) | Flag for the existence of analytics |
| | | | specifications |
| if (ATMD_analytics_exist) { | | | |
| ATMD_analytics_size | | u(16) | Size in number of bytes of analytics |
| | | | specifications |
| ATMD_analytics( ) | | u(v) | Analytics specifications |
| } | | | |
| ATMD_linkages_exist | | u(1) | Flag for the existence of linkage |
| | | | information |
| if (ATMD_linkages_exist) { | | | |
| ATMD_linkages_size | | u(16) | Size in number of bytes of linkage |
| | | | information |
| ATMD_linkages( ) | | u(v) | Linkages to other data objects |
| } | | | |
| ATMD_history_exist | | u(1) | Flag for the existence of access history |
| if (ATMD_history_exist) { | | | |
| ATMD_history_size | | u(16) | Size in number of bytes of access history |
| ATMD_history( ) | | u(v) | Access history of the annotation table |
| } | | | |
| reserved | | u(4) | Trailing zeros for byte alignment |
| } | | | |

TABLE 2
Syntax of Annotation Table Protection

| Syntax | Key | Type | Remarks |
|---|---|---|---|
| annotation_table_protection { | atpr | | |
| dataset_group_ID | | u(8) | Dataset group identifier |
| dataset_ID | | u(16) | Dataset identifier |
| AT_ID | | u(8) | Annotation table identifier |
| AT_protection_value( ) | | | Protection metadata |
| } | | | |

The annotation table is highly configurable. According to an embodiment, the annotation table comprises general metadata that comprises general information about the annotation table. For example, the general metadata may comprise a TableInfo element with information useful for converting and exporting the data of an annotation table to a compatible file format. The general metadata may also comprise TableViewProfile elements for specifying the sets of viewing parameters for individual users or roles. A user can be associated with multiple profiles through their ID and role, with one designated as the default profile. A user can also define their own profiles and share them with other users. Within a view profile, parameters can be specified at three levels, such as common, attribute group-specific, or field-specific parameters. With this hierarchical approach, parameters only need to be specified for a component when they differ from those defined at the upper level. The TableViewProfile element can also include a set of formatting rules for filtering, sorting and highlighting, which are useful for the analysis of annotation table data. Users can share their filtering analyses by making their table view profiles available to other users. Both the TableInfo and TableViewProfile elements can be individually encrypted and signed.

According to an embodiment, the annotation table comprises analytics metadata that comprises pipeline specifications and verification results of data reproducibility. For example, the analytics metadata may comprise pipeline elements for the specification of an analytical pipelines, each of which includes the input data, software tools, processing steps, and mappings of the generated output data to existing data. The analytics metadata may comprise verification elements for the storage of verification results, each of which includes the ID of the pipeline being evaluated, the selected data objects, rules, and status of the verification. Both the pipeline and verification elements can be individually encrypted and signed. The system may therefore comprise an automatic process for the verification of data reproducibility.

According to an embodiment, the annotation table comprises access history metadata that contains secure access history for data traceability or non-repudiable access tracking. The actions that should be recorded for specific data objects and regions can be specified in RecordRule elements. Each AccessRecord element can register the details of a data access, which includes the specific action, the target data objects and regions, the situation (e.g. emergency), any additional notes, the ID and role of the user who performed the action, and the access time, among other possible options. Each AccessRecord element can be signed using the private key of the user who performed the action to ensure the non-repudiation of the action.

According to an embodiment, the annotation table comprises data linkage metadata that comprises specifications of linkages between the annotation table and other data objects for purposes such as data exploration, navigation, visualization, and join query, among other purposes. The data linkage metadata supports mapping by index, where rows/columns of one annotation table can be mapped directly to the rows/columns of another annotation table. The data linkage metadata supports mapping by value, where two annotation tables are linked by some mapping conditions based on the values of specific fields. With linkages properly defined in the metadata, join query on multiple annotation tables is readily supported, and its implementation is explained through an example.

According to an embodiment, each of the metadata components consisting of the entire XML, document can be encrypted and signed with the inclusion of table ID, table name, table version, last update user ID and last update time to increase the uniqueness of the signature value to prevent it from being reused.

Annotation Table Metadata

The structure in which the annotation table metadata is stored may take any of a wide variety of formats. Although a specific format is described with reference to an embodiment, below, it is understood that this is just one example of a data structure that may be utilized by the genomic data storage system described or otherwise envisioned herein.

According to an embodiment, the Annotation Table Metadata gen_info box with key “atmd” consists of four main components: (i) ATMD_general( ) that contains general information about the annotation table; (ii) ATMD_analytics( ) that contains analytics specifications for the verification of data reproducibility; (iii) ATMD_history( ) that contains secure access history for data traceability; and (iv) ATMD_linkages( ) that contains specifications of linkages between the annotation table and other data objects for purposes such as data exploration, navigation, visualization and join query.

According to just one embodiment, each of these components is in the form of an XML, document compressed by the LZMA algorithm. To protect the confidentiality and integrity of a metadata component, which might contain sensitive information, its encryption and signing can be enabled by specifying its URI and relevant parameters in the protection metadata of the same annotation table. With proper access control settings, only authenticated and authorized users can read, update, or sign on the component. If signing is enabled, only the latest signature is kept. To further prevent the metadata component and its corresponding signature from being replaced by an obsolete previous version, optional LastUpdateUser element of type string and LastUpdateTime element of type dateTime can be included in the XML document for encryption and signing, with the corresponding update record, including the last update user and time, entered into ATMD_history( ). Similarly, optional TableID, TableName and TableVersion elements of type string can be included to ensure that the metadata component can only be used for the table of specific ID, name and version. In this case, the metadata component has to be updated with proper encryption and signing whenever the table ID or version is changed.

General Metadata

According to an embodiment, general metadata is used for holding the general information of an annotation table. It is stored in the ATMD_general( ) field as a compressed XML document with a root element “ATMD_General”, which consists of three main components: BasicInfo, TableInfo, and one or multiple instances of TableViewProfile.

According to an embodiment, the BasicInfo element shares the same structure as DatasetGroup and Dataset elements. In general, element values in dataset metadata are inherited by an annotation table within the dataset. However, for each extension element in dataset metadata, its corresponding “Inheritable” element needs to be specified as “true” in order for the extension element value to be inherited by a subordinate annotation table. An element value in BasicInfo overwrites the corresponding element value inherited from the dataset, i.e., the new element value in the general metadata of an annotation table is a specialization of the equivalent element in the metadata of the enclosing dataset.

According to an embodiment, TableInfo contains additional metadata elements specific to annotation tables, which include but are not limited to the following: (i) ImportFileInfo—information of the original file, such as file name, size and number of lines, if the data is imported; (ii) CompatibleFileFormats—any external file formats and their latest versions that are compatible/interconvertible with the annotation table; (iii) Headerlines—any header lines with their line numbers, which could be included with the exported text file; (iv) CommentLines—any comment lines with their line numbers, which could be included with the exported text file; (v) Notes—additional notes; (vi) Correspondence—contact information; (vii) TableCreatedBy—ID of the user who created the annotation table; and/or (viii) TableCreatedTime—date and time of the creation of the annotation table

According to an embodiment, TableViewProfile contains a set of viewing parameters, which include but are not limited to the following attributes and elements: (i) id, name—ID and name of the view profile; (ii) userID—user ID associated with the view profile (if a user is associated with multiple view profiles, then the attribute “profilePriority” specifies the priority of the profile, with 0 indicating it is the default profile for display for that user); (ii) role—user role associated with the view profile (if a user role is associated with multiple view profiles, then the attribute “profilePriority” specifies the priority of the profile, with 0 indicating it is the default profile for display for the user role); (iii) ProfileNotes—notes on the view profile, e.g. to describe its use and purpose; (iv) CommonViewPars—a set of default viewing parameters that apply to all fields. It includes the settings for font, alignment, margins, line spacing, column width, row height, background color, zoom level, indices of the top row and leftmost column for display, selected region, locations of frozen panes, transposition of rows and columns, etc.; (v) AttributeGroupViewPars—a set of viewing parameters specific to fields belonging to the same attribute group.

According to an embodiment, AttributeGroupViewPars can comprise one or more of: agClass—attribute group class to which the parameters apply; hide—boolean value, if true, all fields in the attribute group are hidden from display; and/or location—where to place the group of attributes. For example, attributes associated with the rows of the main table, i.e. attribute group class of 1, can be placed either on the left or on the right of the main attribute group. Similarly, attributes associated with the columns, i.e. attribute group class of 2, can be placed either on the top or bottom of the main attribute group. The main attribute group is always located in the center. AttributeGroupViewPars can also comprise fields, which specify which data fields should be displayed, their order in the presented table, whether a field header should be shown, the field header text and other parameters specific to each field. Note that general display parameters, such as font, alignment, margins, line spacing and background, can be overridden at the attribute group or data field levels.

According to an embodiment, TableViewProfile further comprises: (vi) FormattingRules—a set of formatting rules to be applied on the annotation table. FormattingRules can comprise, for example: FilterRules—each filtering rule specifies the field on which the rule is applied, and the filtering condition; SortRules—each sorting rule specifies the field on which the rule is applied, and the sorting order (ascending or descending); and/or HighlightRules—each highlighting rule specifies the highlighting condition and color. According to an embodiment, TableViewProfile further comprises: (vii) CreatedBy—ID of the user who created the view profile; (viii) CreatedTime—Date and time of the creation of the view profile; and (ix) Signature—a digital signature, with its associated parameters, generated using the private key of the user who created the view profile for proving the authenticity of the set of view parameters and formatting rules.

Analytics Metadata

According to an embodiment, analytics metadata is used for keeping detailed specifications of the software pipelines for generating the data of one or multiple annotation tables. This allows the verification of data reproducibility by re-running the analysis using exactly the same input data, computational environment, software and pipeline settings, and comparing the generated results with the existing annotation table data. The metadata can be further protected by encryption and digital signature, and is stored in the ATMD_analytics( ) field as a compressed XML, document with a root element “ATMD_Analytics”, which contains two main groups of elements: Pipelines and Verifications.

According to an embodiment, each Pipeline element consists of, but is not limited to, one or more of the following attributes and elements: (i) id, version—ID and version of the analytical pipeline; (ii) Tools—a set of software tools used in the pipeline. Each tool is specified by a set of parameters, including a unique tool ID, name and version of the software, source—a URI for obtaining the software and its documentations, description, path—a pointer to an installed copy of the tool, and alias—a shortcut for the tool command. Further: (iii) InputData—one or multiple instances of InData element of DataRefType, each specifying an input data object for the pipeline; (iv) Process—a sequence of processing steps of ProcStepType, each of which includes one or more of: procStepID—a sequential index of the step in the pipeline; ToolID—ID of the software tool used in this step, must be one of the IDs defined in Tools; ToolPars—a string of command line parameters for running the tool. It can contain aliases, prefixed by symbols such as “$”, to be replaced by the paths to the input/output directories/files defined in the InData or OutData elements associated with the step; InDataID—ID that references one of the data objects defined in InputData or the OutData elements of the previous steps; InData—If an input data object has not been previously defined, then an InData element of DataRefType can be specified; OutData—an output data element of DataRefType for specifying the output directory and file.

According to an embodiment, there can be multiple instances of InDataID, InData and OutData if the command line of the tool involves multiple input/output directories or data objects represented by their respective aliases. If both InDataID and InData are not specified, then it is assumed that the input data is from the output data of the previous step.

According to an embodiment, each Pipeline element may consist of, but is not limited to, one or more of the following attributes and elements: (v) OutputDataMaps—one or multiple instances of DataMap element of DataMapType, each mapping a generated output data object to an existing data object. The two data objects are supposed to be equivalent and their contents should therefore be the same or close enough as a proof for the reproducibility of the analytical pipeline. A DataMap element includes one or more of: either GenDataID or GenData—an ID of a previously defined OutData element in the pipeline or a DataRefType element that references a generated output data; ExistData—a DataRefType element that references an existing data object. Each Pipeline element may further comprise, but is not limited to, one or more of the following attributes and elements: (vi) UserID, Role—ID and role of the user who last edited this pipeline specifications; (vii) LastUpdateTime—date and time of the last update to this pipeline specifications; (viii) Signature—a digital signature, with its associated parameters, generated using the private key of the user who last updated the Pipeline element for proving the authenticity of the pipeline specifications

According to an embodiment, regarding DataRefType for the InData and OutData elements in a pipeline, the element type consists of the following attributes and elements: (i) dataRefID—ID of the data reference; (ii) DirURI—a URI that references the directory of the data reference; (iii) Filename—file name of the data reference; (iv) MpggURI—a URI that references a specific data object, such as an annotation table, in the file; (v) NumberCounter—used for generating a sequence of numbers, each of which to be inserted into a URI or file name through its alias prefixed by a symbol such as “$”; (vi) LetterCounter—used for generating a sequence of letters, each of which to be inserted into a URI or file name through its alias prefixed by a symbol such as “$”.

According to an embodiment, there is a one-to-one correspondence of the counter sequences, i.e. the i th sequence value of each of the counters will be inserted together into the i th data reference. As a result, if there are n sequence values for each counter, n data objects will be referenced. For example, the following DataRefType element represented by the alias “inFile” will results in four file names “InFile_A1.dat”, “InFile_A2.dat”, “InFile_B1.dat” and “InFile_B2.dat”, since the generated letter sequence is “AABB” and the generated number sequence nc is “1212”:

```
<InData dataRefID=“InData1”>
  <Filename alias=“inFile”>InFile_${lc}${nc}.dat</Filename>
  <LetterCounter alias= “lc” start=“A” end=“B” repsPerCount=2/>
  <NumberCounter alias= “nc” start=1 end=2 numCounts=4/>
</InData>
```

According to an embodiment, if $ {inFile} is placed in the parameter string of a processing step, such as “−i $ {inFile}”, it will result in the command being executed four times, once for each of files referenced by InData1.

According to an embodiment, each Verification element contains the results of data reproducibility verification that involves running a defined pipeline and comparing the generated data objects with the equivalent existing data objects. It consists of, but is not limited to, one or more of the following attributes and elements: (i) id—ID of the Verification element; (ii) PipelineID—ID of the pipeline being verified; (iii) SelectedDataMaps—one or multiple DataMap IDs defined in the OutputDataMaps element of the pipeline for selecting the pairs of generated and existing data objects for verification. If not specified, all data maps in OutputDataMaps are verified; (iv) VerificationRules—a set of verification rules, each of which includes one or more of: DataMapID—ID of the data map on which the verification rule applies; Attributes—a list of attribute IDs or names in the data objects referenced by DataMapID on which the verification rule applies; Descriptors—a list of descriptor IDs or names in the data objects referenced by DataMapID on which the verification rule applies; DataType—the data type on which the verification rule applies. If DataMapID is specified, the rule is only applicable to the data objects referenced by DataMapID. Otherwise, it generally applies to all data objects of the specified data type; Method—method for evaluating the difference between two data elements, e.g. “number of different entries”, “root mean square”, “sum of absolute differences”, etc.; PassCondition—the pass condition based on the measure generated by the specified method, e.g. “<0.01” means that the measure should be smaller than 0.01 for passing this rule.

According to an embodiment, each Verification element further comprises one or more of the following attributes and elements: (v) Status—status of the verification, e.g. “Pass” or “Fail”; (vi) Platform—a description of the platform on which the verification is performed; (vii) OS—a description of the operating system environment in which the verification is performed; (viii) Notes—additional notes for the verification, e.g. for each pair of data objects being compared, whether they are significantly different and a measure of the difference; (ix) UserID, Role—ID and role of the user who performed the verification; (x) VerificationTime—date and time when the verification was performed; and/or (xi) Signature—a digital signature, with its associated parameters, generated using the private key of the user who performed the verification for proving the authenticity of the verification results.

According to an embodiment, with all details of the pipeline and the verification rules specified, automatic verification of data reproducibility can be performed. The verification process should include the following steps: (1) Check whether or not all the input data objects, and existing data objects defined in the selected data maps are available; (2) Check whether or not all the required software tools are properly installed with the right version; (3) Check the correctness of the process specifications, e.g. the input data objects for each step must link to existing data objects or output data objects defined in previous steps; (4) Check whether or not the verification rules cover all attributes and descriptors in selected data maps. A scheduler and despatcher should execute the processing steps one after another, i.e. only execute a step when all input data objects supposed to be generated from the previous steps are available. If a step has multiple sets of input files (defined using number and string counters), the software tool can be run on each set of input files in parallel. Verification of a generated data object defined in SelectedDataMap can be performed as soon as it becomes available. For each attribute/descriptor, identify the right verification rule(s) by looking up the data map ID and attribute/descriptor name/ID. If there is no specific rule for the attribute/descriptor, look up any rule(s) associated with the data map ID and the data type of the attribute/descriptor. If that is not available, then look up the rule for the data type that generally applies to all data objects. After identifying the right rules for all the attributes and descriptors in the data object, evaluate the difference of each attribute/descriptor between the generated and existing data using the methods defined in the applicable verification rules. A data object passes the verification only if all its attributes/descriptors satisfy the pass conditions in the applicable verification rules.

According to an embodiment, after completing the execution of all processing steps and the verifications of all data objects in selected data maps, the pipeline being verified for reproducibility can be assigned a “Pass” status if all the generated data objects pass their verifications. The verification results can then be signed using the private key of the user who performed the verification and stored as a Verification element in the metadata. Note that the process should stop if it does not pass any one of the first four checking steps.

Access History Metadata

According to an embodiment, access history metadata is used for registering selected user actions, such as viewing or changing any metadata elements or annotation table data, with support for digital signatures to ensure data traceability or non-repudiable access tracking. It is stored in the ATMD_history( ) field as a compressed XML document with a root element “ATMD_History”, which contains two main groups of elements: RecordRules and AccessRecords.

According to an embodiment, each RecordRule element specifies the user actions that should be recorded for specific data objects or regions. If there is no RecordRule element, then all actions on all data should be recorded. A RecordRule element comprises, but is not limited to, one or more of the following attributes and elements: (i) id—ID of the record rule; (ii) Actions—an element for specifying the actions to be recorded. Its status attribute first determines if all actions should be included or excluded to begin with. If the status is “Include All”, then its enclosed Action elements are to be excluded. On the contrary, if the status is “Exclude All”, then all its enclosed Action elements are to be included; (iii) TargetURI—a URI that references the data object, e.g. a metadata component or the protection metadata, on which the rule applies; (v) TargetRegion—a set of elements specifying the annotation table data on which the rule applies. The first group of elements: “AttributeGroups”, “Attributes” and “Descriptors” concerns the selection of attributes and descriptors through their IDs, names or affiliated attribute groups. The second group of elements “GenomicRanges”, “SampleRanges”, “RowRanges” and “ColRanges” concerns the selection of rows and columns in the table through a combination of ranges based on genomic coordinates, sample IDs, row indices and column indices.

Note that if no TargetURI or TargetRegion element is specified, then the selected actions are recorded on all data. For target data overlapped by multiple record rules, the actions to be recorded in that target should be a union of the selected actions in those rules.

According to an embodiment, each AccessRecord element registers the details of a data access action. It comprises, but is not limited to, one or more of the following attributes and elements: (i) id—ID of the access record, could be a sequential index; (ii) Action—a string that specifies the specific action, which could be the name of a function call, being performed and registered; (iii) TargetURI—a URI that references the data object, e.g. a metadata component or the protection metadata, on which the action was performed; (iv) TargetRegion—a set of elements specifying the annotation table data on which the action was performed. The first group of elements: “AttributeGroups”, “Attributes” and “Descriptors” concerns the selection of attributes and descriptors through their IDs, names or affiliated attribute groups. The second group of elements “GenomicRanges”, “SampleRanges”, “RowRanges” and “ColRanges” concerns the selection of rows and columns in the table through a combination of ranges based on genomic coordinates, sample IDs, row indices and column indices; (v) Situation—a string that indicates the situation under which the action was performed, e.g. “Emergency”; (vi) Notes—additional notes on the action; (vii) UserID, Role—ID and role of the user who performed the action; (viii) AccessTime—date and time when the action was performed; and/or (ix) Signature—a digital signature, with its associated parameters, for the access record to prove its authenticity. To ensure non-repudiation, it should be generated using the private key of the user who performed the action.

The process for verifying the integrity of access history can include the following steps: (1) Check whether or not the IDs of the access records are in consecutive increasing order; (2) Check whether or not the access time of the access records are in chronological order; (3) Check whether or not the table ID, table name and table version appended to the history are the same as the ones currently in use; (4) Verify the digital signatures of all access records; (5) Verify the digital signature of the whole access history metadata ATMD_history( ). The verification is successful only if it passes all individual steps.

Data Linkage Metadata

According to an embodiment, data linkage metadata is used for specifying any relationships that exist between the current annotation table and other data objects within or without the current file archive in order to facilitate cross-referencing capabilities for purposes such as data exploration, navigation, visualization and join query. It is stored in the ATMD_linkages( ) field as a compressed XML document with a root element “ATMD_Linkages”, which can contain more than one set of parameters for specifying the linkages with other data objects, such as a bam file, a dataset of sequencing reads or an annotation table.

According to an embodiment, each linkage definition comprises, but is not limited to, one or more of the following attributes and elements: (i) id—an identifier of the Linkage element unique within the XML document; (ii) Description—a text description of the linkage being defined; (iii) Alias—a name for uniquely identifying the linked data object, e.g. to be used in SQL join queries. If not specified, then the name of the linked data object should be used; and/or (iv) URI reference to the linked object consisting of at least one of: FileURI—a URI for referencing the file that is linked. If not specified, the linked object is in the same file as the current annotation table; MpggURI— a URI for referencing the specific MPEG-G data object within the file. If not specified, the linkage is to the whole file. In general, the URI follows the format:
“dataset_group/{dataset_group_id}/dataset/{dataset_id}/ann_table/{ann_table_tag}”
   where the text within curly brackets, including the curly brackets themselves, shall be replaced by the IDs (number fields) or names (string fields) of the dataset group, dataset and annotation table being referenced. In cases where the same tag is used for the ID of one object and the name of another object, then the one with the matching ID is referenced. Contraction of the URI is allowed by omitting the beginning parts that are the same as the current annotation table. For example, if the URI is referencing another annotation table in the same dataset, then it can be simplified as “ann_table/{ann_table_tag}”. If the referenced object is a dataset, then the part “/ann_table/{ann_table_tag}” can be omitted. If the linked object is an annotation table, one can further specify how the current annotation table can be mapped to the linked table. If the rows/columns of the current annotation table is directly mapped to the rows/columns of another table, then the MapByIndex element should be included with a “method” attribute that can only assume one of the four values: “row-to-row”, “row-to-col”, “col-to-row” and “col-to-col”.

According to an embodiment, if the current annotation table is mapped to another table by matching some attribute values, then the MapByValue element should be included to specify one or more mapping conditions joined by “AND” operators by default. Each of the conditions can comprise one or more of: relation_op—a relational operator, which can be “=”, “<”, “<=”, “>”, “>=” or “!=”, between FromField on the left and ToField on the right; FromField—a URI for referencing the descriptor or attribute of the current annotation table. Its possible formats include “descriptor/{desc_tag}” and “attribute/{attr_tag}”, where the text within curly brackets, including the curly brackets themselves, shall be replaced by the id (a number field) or name (a string field) of the descriptor/attribute used in the mapping. In cases where the same tag is used for the ID of one object and the name of another object, then the one with the matching ID is referenced; and/or ToField—a URI for referencing the descriptor or attribute of the linked annotation table. Its possible formats are the same as those of FromField.

One non-limiting example is to link an annotation table containing the variant calls of a single sample to its source sequencing-read dataset. Suppose both entities are in the same dataset group of an MPEG-G file, with the sequencing reads in the dataset of ID 1 and the variant calls in the dataset of ID 2. The linkage can then be defined in the metadata of the variant-call annotation table, with an optional linkage ID “SeqReadLinkage” and MpggURI set to “dataset/1”. With this linkage defined, the sequencing reads associated with any variant of interest can be looked up by genomic position to provide the supporting evidence for the variant call as needed by a user.

Another example is to use data linkages for join queries. Suppose a genomic study consists of the following annotation tables within the same MPEG-G dataset: (i) a gene expression table named “GeneExpr”, which rows uniquely identified by “gene_symbol” attribute and columns uniquely identified by “sample_ID” attribute; (ii) a gene information table named “GeneInfo” containing additional annotations, such as chromosome, start and end positions, and known disease associations for each gene, with rows uniquely identified by “gene_entrez_ID” attribute; (iii) a table “GeneIdMap” that provides the mapping between “gene_symbol” and “gene_entrez_ID”; and (iv) a sample information table named “SampleInfo” containing additional demographic and clinical data, such as gender, age, ethnicity and diagnosis for each sample, with rows uniquely identified by “sample_ID” attribute. The following data linkages can then be defined: (i) In the ATMD_Linkages( ) field of the metadata of the table GeneExpr: a linkage of ID “EntrezIdLinkage”, with MpggURI=“ann_table/GeneIdMap”, and a MapByValue element with relation_op=“=”, FromField=“attribute/gene_symbol” and ToField=“attribute/gene_symbol”; and a linkage of ID “SampleInfoLinkage”, with MpggURI=“ann_table/SampleInfo”, and a MapByValue element with relation_op=“=”, FromField=“attribute/sample_ID” and ToField=“attribute/sample_ID”. Then (ii) in the ATMD_Linkages( ) field of the metadata of the table GeneIdMap, a linkage of ID “GeneInfoLinkage”, with MpggURI=“ann_table/GeneInfo”, and a MapByValue element with relation_op=“=”, FromField=“attribute/gene_entrez_ID” and ToField=“attribute/gene_entrez_ID”.

With the above data linkages defined, a join query can then be performed on the three tables, for example, to select: (1) genes only in the human MHC region located at position 28,477,797-33,448,354 on chromosome 6 (human reference genome GRCh37) and packed with immunity-related genes, and (2) samples of Caucasian ethnicity. The syntax of the query can be something like “SELECT *, GeneldMap.GeneInfo.*, SampleInfo.(Age, Diagnosis) FROM GeneExpr WHERE GeneIdMap.GeneInfo.(Chr=‘6’ AND Start_Pos >=28477797 AND End Pos <=33448354), Samplelnfo.Ethnicity=‘Caucasian’”.

The processing of this query involves two parts: search for genes by genomic range and search for samples by ethnicity. For the gene search, a query engine should first look up the Entrez IDs of the genes in the specified genomic range from the GeneInfo table, then map them to the corresponding gene symbols through the GeneIdMap table and subsequently find the rows in the GeneExpr table associated with the gene symbols. For the sample search, a query engine should first look up the IDs of the samples of Caucasian ethnicity and then find the columns in the GeneExpr table associated with the sample IDs. The query results should include the expression data extracted from the matching rows and columns of the GeneExpr table, the information of the matching genes from the GeneInfo table, and the age and diagnosis of the matching samples from the SampleInfo table.

In addition to join query, data linkages can also facilitate data exploration and navigation. With reference to the linkage example above, an application that presents the gene expression data can allow users to have quick access to the additional information of any genes or samples by clicking or hovering on the gene symbols or sample IDs.

Referring to FIG. 2, in one embodiment, is a schematic representation of a system 200 for storing genomic data. System 200 may be any of the systems described or otherwise envisioned herein, and may comprise any of the components described or otherwise envisioned herein.

According to an embodiment, system 200 comprises one or more of a processor 220, memory 230, user interface 240, communications interface 250, and storage 260, interconnected via one or more system buses 212. In some embodiments, the hardware may include a genomic data database 270. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (MC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, storage 260 of system 200 may store one or more algorithms and/or instructions to carry out one or more functions or steps of the methods described or otherwise envisioned herein. For example, processor 220 may comprise one or more of information metadata generation instructions 262, compression/decompression instructions 263, and/or storage instructions 264.

According to an embodiment, information metadata generation instructions 262 direct the system to generate or modify an information metadata structure within the file structure for the genomic dataset. The information metadata structure is configured to enable a wide variety of functionalities, including one or more of support for selective encryption and digital signatures, data traceability or non-repudiable access tracking, verification of data reproducibility, and establishment of linkages between data objects, among other functionalities. According to an embodiment, the annotation table comprises one or more of: (i) information about an annotation table, including one or more user profiles and associated profile permissions; (ii) analytics information detailing a source dataset and one or more processing steps for producing the genomic dataset, wherein the analytics information is configured to facilitate verification of data reproducibility; (iii) access history for the genomic dataset, configured to facilitate data traceability; and/or (iv) linkage information defining a relationship between the annotation table and one or more data objects, wherein the linkage information is configured to enhance data navigation and/or to support and data query across linked data.

According to an embodiment, compression/decompression instructions 263 direct the system to compress the genomic data as well as the associated information metadata structure. The compression algorithm can be any algorithm, method, or process for data compression. The compression instructions may also comprise decompression instructions for decompression stored data. The compression/decompression instructions may comprise one compression and/or decompression algorithm, or may comprise a plurality of compression and/or decompression algorithms.

According to an embodiment, storage instructions 264 direct the system to store the compressed genomic data and compressed information metadata in a container data structure. The system may comprise or be in communication with local or remote data storage configured to store the genomic dataset and information metadata.

The processing of a genomic dataset, the generation of an information metadata structure, and compression/decompression of the genomic data and information metadata structure comprises millions or billions of calculations, something the human mind is not equipped to perform, even with pen and pencil. Indeed, the genomic dataset alone comprises millions of pieces of information. For example, next-generation DNA sequencing data comprises reads that number in the 100s of millions or even billions.

Further, the methods described herein significantly improve the speed and functionality of a genomic storage system. For example, by implementing the methods described herein, the genomic storage system comprises an information metadata structure that includes: (i) information about an annotation table within the file structure, including one or more user profiles and associated profile permission; (ii) analytics information detailing a source dataset and one or more processing steps for product the genomic dataset, wherein the analytics information is configured to facilitate verification of data reproducibility; (iii) access history for the genomic dataset, configured to facilitate data traceability; and (iv) linkage information defining a relationship between the annotation table and one or more data objects, wherein the linkage information is configured to enhance data navigation and/or to support a data query across linked data. Prior art systems cannot provide this functionality, and therefore are inferior systems. Accordingly, the methods described herein significantly improve the speed and functionality of a genomic storage system.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for storing genomic data within a data structure comprising a file structure, the method comprising:
receiving a genomic dataset comprising a plurality of fields or attributes of different data types;
generating an information metadata structure for the genomic dataset, comprising one or more of: (i) information about an annotation table within the file structure, including one or more user profiles and associated profile permission; (ii) analytics information detailing a source dataset and one or more processing steps for producing the genomic dataset, wherein the analytics information is configured to facilitate verification of data reproducibility; (iii) access history for the genomic dataset, configured to facilitate data traceability; and (iv) linkage information defining a relationship between the annotation table and one or more data objects, wherein the linkage information is configured to enhance data navigation and/or to support a data query across linked data;
compressing the genomic data, and the information metadata, using one or more compression algorithms to generate a compressed genomic dataset and compressed information metadata; and
storing the compressed genomic dataset and the compressed information metadata in a container data structure;
wherein some or all of the annotation table is encrypted.

2. The method of claim 1, further comprising the steps of:
receiving new data for the annotation table; and
updating the annotation table, comprising updating one or both of the information metadata and the genomic data.

3. The method of claim 1, wherein one or more of (i) through (iv) comprise selective encryption and a digital signature.

4. The method of claim 1, wherein the access history for the genomic dataset is configured to track access and/or change to the genomic data by one or more users, and wherein tracked access or changes are predefined.

5. The method of claim 4, wherein the access history further comprises an identity of a user that accessed the genomic data and/or made a change to the genomic data, and wherein the access history optionally comprises an accompanying digital signature of the user.

6. The method of claim 1, wherein the one or more user profiles comprise one or more parameters for presentation and/or further processing such as filtering, sorting, and/or highlighting of the genomic data.

7. The method of claim 1, wherein the one or more user profiles can be created by a user, encrypted for confidentially, signed for authenticity, and/or shared with another designated user.

8. The method of claim 1, wherein the analytics information comprises instructions for verification of data reproducibility by evaluating a concordance of the genomic dataset with an existing counterpart genomic dataset being verified.

9. The method of claim 1, wherein the analytics information further comprises one or more verification results, with an optional digital signatures by a user that performed the verification.

10. The method of claim 1, wherein the linkage information comprises one or more specifications for mapping data between one or more annotation tables.

11. The method of claim 1, further comprising verifying data reproducibility using one or more of: (i) the analytics information and (ii) authenticity and/or integrity of the access history.

12. A system (200) for storing genomic data within a data structure comprising a file structure, the system comprising:
a genomic dataset comprising a plurality of fields or attributes of different data types;
a container data structure configured to store compressed genomic data and compressed information metadata;
a data compression algorithm; and
a processor configured to: (i) generate an information metadata structure for the genomic dataset, comprising one or more of: (1) information about an annotation table within the file structure, including one or more user profiles and associated profile permission; (2) analytics information detailing a source dataset and one or more processing steps for producing the genomic dataset, wherein the analytics information is configured to facilitate verification of data reproducibility; (3) access history for the genomic dataset, configured to facilitate data traceability; and (4) linkage information defining a relationship between the annotation table and one or more data objects, wherein the linkage information is configured to enhance data navigation and/or to support a data query across linked data; (ii) compress, using the data compression algorithm, the genomic data and the information metadata to generate a compressed genomic dataset and compressed information metadata; and (iii) store the compressed genomic dataset and the compressed information metadata in a container data structure;
wherein some or all of the annotation table is encrypted.

13. The system of claim 12, wherein the processor is further configured to: receive new data for the annotation table; and update the annotation table with the new data, comprising updating one or both of the information metadata and the genomic data.

14. The system of claim 12, wherein the analytics information comprises instructions for verification of data reproducibility by evaluating a concordance of the genomic dataset with an existing counterpart genomic dataset being verified.

15. The system of claim 12, wherein the linkage information comprises one or more specifications for mapping data between one or more annotation tables.
